(19) United States
(12) Patent Application Publication
(10) Pub. No.: US 2022/0081374 A1
(43) Pub. Date: Mar. 17, 2022

(54) BUTADIENE PRODUCTION METHOD

(71) Applicants: ENEOS Corporation, Tokyo (JP); JSR CORPORATION, Tokyo (JP)

(72) Inventors: Sosuke HIGUCHI, Tokyo (JP); Nobuhiro KIMURA, Tokyo (JP); Mayu SUGIMOTO, Tokyo (JP); Junjie WANG, Tokyo (JP)

(73) Assignees: ENEOS Corporation, Tokyo (JP); JSR CORPORATION, Tokyo (JP)

(21) Appl. No.: 17/414,137

(22) PCT Filed: Dec. 18, 2019
(86) PCT No.: PCT/JP2019/049703

§ 371 Date: Jun. 15, 2021

(30) Foreign Application Priority Data
Dec. 18, 2018 (JP) 2018-236410

(51) Int. Cl. C07C 5/333

(52) U.S. Cl. CPC C07C 5/3335; C07C 2523/31

(57) ABSTRACT

A method for producing butadiene comprises a step of supplying a raw material gas containing 2-butene and an oxygen-containing gas containing molecular oxygen to a reactor filled with a catalyst to obtain a produced gas containing butadiene, wherein the catalyst contains a composite oxide containing molybdenum and bismuth, and a proportion of cis-2-butene in 2-butene in the raw material gas is 30 to 90 mol %.

### TECHNICAL FIELD

The present invention relates to a method for producing butadiene.

### BACKGROUND ART

Conventionally, a method for producing butadiene through an oxidative dehydrogenation reaction of straight-chain butene in the presence of a catalyst has been known (for example, Patent Literatures 1 and 2).

### CITATION LIST

### Patent Literature

Patent Literature 1: Japanese Unexamined Patent Publication No. S60-115532

Patent Literature 2: Japanese Unexamined Patent Publication No. S60-126235

### SUMMARY OF INVENTION

### Technical Problem

Along with increased demand for butadiene, it is required to develop various butadiene production methods that differ in the required characteristics of production equipment, operating costs, reaction efficiency, etc.

An object of the present invention is to provide a novel method for producing butadiene, enabling efficiently producing butadiene from 2-butene.

### Solution to Problem

One aspect of the present invention relates to a method for producing butadiene, comprising a step of supplying a raw material gas containing 2-butene and an oxygen-containing gas containing molecular oxygen to a reactor filled with a catalyst to obtain a produced gas containing butadiene. In the production method, the catalyst contains a composite oxide containing molybdenum and bismuth. The proportion of cis-2-butene in 2-butene in the raw material gas is 30 to 90 mol %.

In the production method described above, butadiene can be efficiently obtained by using a specific catalyst and 2-butene having a proportion of cis-2-butene within a specific range as raw material. With a proportion of cis-2-butene of less than 30 mol %, the selectivity of butadiene and the yield of butadiene decrease, and for 2-butene with a proportion of cis-2-butene of more than 90 mol %, raw material procurement becomes difficult, so that the efficiency of the whole process decreases.

In an embodiment, the proportion of cis-2-butene in 2-butene in the raw material gas may be 35 to 45 mol %.

In an embodiment, the produced gas may further contain 2-butene, and a proportion of cis-2-butene in 2-butene in the produced gas may be 28 to 50 mol %.

In an embodiment, the proportion of cis-2-butene in 2-butene in the produced gas may be 28 to 32 mol %. By adjusting the reaction conditions of the oxidative dehydrogenation reaction to have such a proportion, the selectivity of butadiene and the yield of butadiene tend to be further improved.

A production method in an embodiment may further comprise a step of contacting a raw material composition containing 1-butene with an isomerization catalyst to isomerize at least a part of 1-butene to obtain 2-butene.

### Advantageous Effect of Invention

According to the present invention, a method for producing butadiene enabling efficiently production of butadiene from 2-butene is provided as a novel production method of butadiene.

### DESCRIPTION OF EMBODIMENTS

Preferred embodiments of the present invention are described as follows.

The method for producing butadiene in the present embodiment comprises a step of supplying a raw material gas containing 2-butene and an oxygen-containing gas containing molecular oxygen to a reactor filled with a catalyst to obtain a produced gas containing butadiene. In the present embodiment, the catalyst contains a composite oxide containing molybdenum and bismuth. The proportion of cis-2-butene in 2-butene in the raw material gas is 30 to 90 mol %.

In the production method, butadiene can be efficiently obtained by using a specific catalyst and 2-butene having a proportion of cis-2-butene within a specific range as raw material. Incidentally, with a proportion of cis-2-butene of less than 30 mol %, the selectivity of butadiene and the yield of butadiene decrease, and for 2-butene with a proportion of cis-2-butene of more than 90 mol %, raw material procurement becomes difficult, so that the efficiency of the whole process decreases. Specifically, for example, in order to obtain 2-butene having a high cis ratio (more than 90 mol %) from a mixed C4 fraction derived from a refined petroleum product, a large amount of energy is required in a distillation column.

The raw material gas may contain straight-chain butene (1-butene and 2-butene) as a main component. The content proportion of straight-chain butene in the raw material gas may be, for example, 50 mol % or more, preferably 60 mol % or more, more preferably 70 mol % or more. The upper limit of the content proportion of straight-chain butene in the raw material gas is not particularly limited and may be 100 mol %.

It is preferable that the raw material gas contain 2-butene as a main component. The content proportion of 2-butene in the raw material gas may be, for example, 50 mol % or more, preferably 60 mol % or more, more preferably 70 mol % or more. The upper limit of straight-chain butene content proportion in the raw material gas is not particularly limited and may be 100 mol %.

The 2-butene in the raw material gas may include cis-2-butene and trans-2-butene. The proportion X₁ of cis-2-butene in 2-butene in the raw material gas is 30 mol % or more, preferably 35 mol % or more, more preferably 37 mol % or more. Further, the proportion X₁ is 90.0 mol % or less, and from the viewpoint of easily obtaining a raw material from a mixed C4 fraction derived from a refined petroleum product at low cost, preferably 70 mol % or less, more preferably 60 mol % or less.

The raw material gas may further contain components other than straight-chain butene. For example, the raw material gas may further include butane. Examples of the butane include n-butane and isobutane. The content proportion of butane in the raw material gas is not particularly limited and may be, for example, 50 mol % or less, preferably 40 mol % or less, more preferably 30 mol % or less.

It is preferable that the concentration of isobutene in the raw material gas be low, and the concentration may be, for example, 3 mol % or less, preferably 1 mol % or less.

It is preferable that the concentration of butadiene in the raw material gas be low, and the concentration may be, for example, 3 mol % or less, preferably 1 mol % or less.

The raw material gas may further contain a hydrocarbon having 5 or more carbon atoms. In a method for producing butadiene, a by-product (or a polymer produced from the by-product, and the like) may be deposited in the latter stage of a reactor to cause clogging of the reactor in some cases. Due to the raw material gas containing a hydrocarbon having 5 or more carbon atoms, when the produced gas is cooled in the latter stage of a reactor, the hydrocarbon having 5 or more carbon atoms condenses into liquid to dissolve or wash away the by-product, so that clogging of the reactor can be prevented. From the viewpoint of remarkably obtaining the effect, the content proportion of the hydrocarbon having 5 or more carbon atoms in the raw material gas is preferably 0.05 mol % or more, more preferably 0.1 mol % or more, still more preferably 0.2 mol % or more. From the viewpoint of reaction efficiency, the content proportion of the hydrocarbon having 5 or more carbon atoms in the raw material gas is preferably 7 mol % or less, more preferably 6 mol % or less, still more preferably 5.5 mol % or less.

The hydrocarbon may have, for example, 25 or less, preferably 20 or less, more preferably 15 or less carbon atoms. Although the hydrocarbon is not particularly limited, a saturated hydrocarbon is preferred. Further, the hydrocarbon may be in a straight-chain form, a branched-chain form, or a cyclic form. A straight-chain form or a branched-chain form is preferred.

As the raw material gas, for example, a fraction containing a straight-chain butene and butanes obtained by separating butadiene and isobutene from a C4 fraction by-produced in naphtha decomposition may be used. Alternatively, as the raw material gas, for example, a fraction produced by a dehydrogenation reaction of n-butane may be used. Alternatively, as the raw material gas, for example, a fraction obtained by dimerization of ethylene may be used. Alternatively, as the raw material gas, for example, a C4 fraction obtained from fluid catalytic cracking may be used. In the fluid catalytic cracking, the C4 fraction is obtained by decomposing a heavy oil fraction obtained in distillation of crude oil in an oil refinery plant or the like using a powdery solid catalyst in a fluidized bed state so as to be converted into a low boiling-point hydrocarbon.

Alternatively, the raw material gas may be one obtained by isomerization reaction of the fraction described above so as to increase the proportion of 2-butene from that of the fraction through isomerization of at least a part of 1-butene. In other words, the production method of the present embodiment may further comprise a step of contacting a raw material composition containing 1-butene with an isomerization catalyst to isomerize at least a part of 1-butene to obtain 2-butene.

The isomerization catalyst and the reaction conditions for the isomerization reaction are not particularly limited, and known catalysts and conditions capable of isomerizing 1-butene to 2-butene may be used without particular limitation. The isomerization catalyst may include, for example, at least one selected from the group consisting of silica, alumina, silica-alumina, zeolite, activated clay, diatomaceous earth, and kaolin. Alternatively, the isomerization catalyst may include at least one selected from the group consisting of silica and alumina. Alternatively, the isomerization catalyst may be composed of silica alumina.

Further, the isomerization catalyst may have a carrier and an element supported on the carrier (hereinafter, referred to as “supported element” in some cases). The carrier may include, for example, at least one selected from the group consisting of silica, alumina, silica-alumina, zeolite, activated carbon, activated clay, diatomaceous earth and kaolin.

Alternatively, the carrier may include at least one selected from the group consisting of silica and alumina, or may be composed of zeolite.

The supported element of the isomerization catalyst may be, for example, at least one element selected from the group consisting of elements in group 10 in the periodic table, elements in group 11 in the periodic table, and lanthanoids. The periodic table refers to the long period type periodic table of elements based on the IUPAC (International Union of Pure and Applied Chemistry) rules. The supported element may be an element other than group 10 elements in the periodic table, group 11 elements in the periodic table, and lanthanoids. The group 10 element in the periodic table may be, for example, at least one selected from the group consisting of nickel (Ni), palladium (Pd), and platinum (Pt). The group 11 element in the periodic table may be, for example, at least one selected from the group consisting of copper (Cu), silver (Ag), and gold (Au). The lanthanoid may be, for example, at least one selected from the group consisting of lanthanum (La) and cerium (Ce). The elements supported on the carrier may be a combination of these elements. It is preferable that the element supported on the carrier be Ag.

The reaction conditions for the isomerization reaction are not particularly limited, and for example, the reaction temperature may be 150 to 450° C., preferably 250 to 400° C., more preferably 300 to 380° C. Further, the gas space velocity (GHSV (h⁻¹)) of the raw material straight-chain butene maybe, for example, 0.01 to 50.0 h⁻¹, preferably 0.05 to 10.0 h⁻¹.

In the present embodiment, the reactor used for the oxidative dehydrogenation reaction is not particularly limited. Examples of the reactor include a tubular reactor, a tank reactor, and a fluidized bed reactor. The reactor is preferably a fixed bed reactor, more preferably a fixed bed multi-tubular reactor. These reactors may be those generally industrially used.

The oxygen-containing gas maybe, for example, a gas containing 10 vol % or more of molecular oxygen (O₂), preferably a gas containing 15 vol % or more of molecular oxygen, more preferably 20 vol % or more of molecular oxygen. The oxygen-containing gas may be, for example, air. From the viewpoint of cost reduction, the concentration of molecular oxygen in the oxygen-containing gas may be 50 vol % or less, preferably 30 vol % or less, more preferably 25 vol % or less.

The oxygen-containing gas may contain components other than molecular oxygen within the range in which the effect described above is exhibited. Examples of the component include nitrogen, argon, neon, helium, CO, CO₂, and water. The concentration of nitrogen (molecular nitrogen) in the oxygen-containing gas may be, for example, 50 vol % or more, 70 vol % or more, or 75 vol % or more. The concentration of nitrogen in the oxygen-containing gas may be, for example, 90 vol % or less, 85 vol % or less, or 80 vol % or less. The concentration of the components other than nitrogen may be, for example, 10 vol % or less, preferably 1 vol % or less.

In supplying of the raw material gas to the reactor, nitrogen gas and water (steam) may be supplied together with the raw material gas and the oxygen-containing gas. Nitrogen gas is supplied from the viewpoint of adjusting the concentrations of the combustible gas and the molecular oxygen, such that the reactant gas does not form a detonating gas. Water (steam) is supplied from the viewpoint of adjusting the concentrations of the combustible gas and the molecular oxygen as in the case of nitrogen gas, and the viewpoint of suppressing coking of the catalyst.

As a result of mixing between the raw material gas and the oxygen-containing gas, a mixture of combustible gas and molecular oxygen is formed. Accordingly, the composition at the inlet of the reactor may be controlled through monitoring of the flow rate of each gas (raw material gas and oxygen-containing gas, and on an as needed basis, nitrogen gas and water (steam)) with a flow meter installed in a pipe for supply, such that the mixture does not falls within the explosive range. By the composition control, for example, the composition range is adjusted to the reactant gas composition described below.

Incidentally, the explosive range is a range in which the mixed gas of combustible gas and molecular oxygen has a composition that ignites in the presence of an ignition source. With a concentration of combustible gas of lower than a certain value, no ignition occurs even in the presence of an ignition source. This concentration is called the lower explosion limit. Similarly, with a concentration of combustible gas of higher than a certain value, no ignition occurs even in the presence of an ignition source. This concentration is called the upper explosion limit. The respective values depend on the oxygen concentration. Generally, the lower the oxygen concentration is, the closer the respective values are, and at an oxygen concentration of a certain value, both coincide. The oxygen concentration on this occasion is called the limiting oxygen concentration, and with an oxygen concentration of lower than this, the mixed gas does not ignite regardless of the concentration of the combustible gas.

In the present embodiment, the following method for initiating the reaction may be employed. For example, the amounts of oxygen-containing gas, nitrogen, and steam initially supplied to a reactor are adjusted, such that the oxygen concentration at the reactor inlet becomes equal to or lower than the limiting oxygen concentration, and then the supply of the raw material gas is initiated. Subsequently, the supply amounts of the raw material gas and the oxygen-containing gas are increased, such that the concentration of combustible gas becomes higher than the upper explosion limit. Alternatively, when the supply amounts of the raw material gas and the oxygen-containing gas are increased, the amounts of nitrogen and/or steam supplied may be reduced, such that the amount of the gas supplied becomes constant. Thereby, the residence time of gas in the pipe and the reactor can be kept constant, so that the fluctuation of pressure can be suppressed.

A typical composition of the reactant gas supplied to a reactor is shown below.

### <Reactant Gas Composition>

Hydrocarbons having 4 carbon atoms: 5 to 15 vol % based on the total amount of reactant gas

Straight-chain butene: 50 to 100 vol % based on the total of hydrocarbons having 4 carbon atoms

O₂: 40 to 120 vol % based on the total of hydrocarbons having 4 carbon atoms

N₂: 500 to 1000 vol % based on the total of hydrocarbons having 4 carbon atoms

H₂O: 90 to 900 vol % based on the total of hydrocarbons having 4 carbon atoms

The reactor is filled with a catalyst described below, and straight-chain butene reacts with oxygen on the catalyst to produce butadiene and water. The oxidative dehydrogenation reaction is an exothermic reaction, and the temperature rises due to the reaction. It is preferable that the reaction temperature be adjusted within the range of 280 to 400° C. It is, therefore, preferable that the reactor be capable of controlling the temperature of the catalyst layer at a constant level by using a heating medium (for example, dibenzyltoluene, nitrite, etc.).

The pressure of the reactor is not particularly limited. The pressure of the reactor is usually 0 MPaG or more, and may be 0.001 MPaG or more, or 0.01 MPaG or more. An increase in the pressure of the reactor provides a merit of enabling supply of a larger amount of the reactant gas to the reactor. On the other hand, the pressure of the reactor is usually 0.5 MPaG or less, and may be 0.3 MPaG or less, or 0.1 MPaG or less. With decrease in the pressure of the reactor, the explosion range tends to be narrowed.

The residence time in the reactor is not particularly limited. The residence time in the reactor may be, for example, 0.1 seconds or more, preferably 0.5 seconds or more. The increase in the residence time value in the reactor provides a merit of increasing the conversion rate of straight-chain butene in the oxidative dehydrogenation reaction. On the other hand, the residence time in the reactor may be, for example, 10 seconds or less, and preferably 5 seconds or less. The smaller the residence time value in the reactor, the smaller the reactor can be.

In the present embodiment, a produced gas containing butadiene is obtained through the oxidative dehydrogenation reaction.

In the present embodiment, the conversion rate of straight-chain butene in the oxidative dehydrogenation reaction may be, for example, 60% or more, preferably 70% or more, and more preferably 80% or more.

The conversion rate of straight-chain butene may be, for example, 99% or less, preferably 95% or less.

With a conversion rate of straight-chain butene of less than 100%, the produced gas further contains straight-chain butene. On this occasion, the produced gas may contain 2-butene.

The proportion X₂ of cis-2-butene in 2-butene in the produced gas is preferably 28 to 50 mol %, more preferably 28 to 32 mol %. By adjusting the reaction conditions and the like of the oxidative dehydrogenation reaction so that the above proportion X₂ in the product gas falls within the above range, the selectivity of butadiene and the yield of butadiene tend to further improve.

The produced gas may contain by-products in the oxidative dehydrogenation reaction. Examples of the by-products include aldehydes. Further, in the case where the raw material gas contains a hydrocarbon having 5 or more carbon atoms, the produced gas may further contain a hydrocarbon having 5 or more carbon atoms.

[Catalyst]

A preferred embodiment of the catalyst (oxidation dehydrogenation reaction catalyst) for use in the production method of the present embodiment is described in detail as follows.

In the present embodiment, the oxidative dehydrogenation reaction catalyst may be a composite oxide catalyst containing a composite oxide containing molybdenum and bismuth.

The composite oxide catalyst may further contain, for example, cobalt.

The composite oxide catalyst may include, for example, a composite oxide represented by the following formula (1).

(Mo)a(Bi)b(Co)c(Ni)d(Fe)e(X)f(Y)g(Z)h(Si)i(O)j (1)

wherein, X represents at least one element selected from the group consisting of magnesium (Mg), calcium (Ca), zinc (Zn), cerium (Ce), and samarium (Sm); Y represents at least one element selected from the group consisting of sodium (Na), potassium (K), rubidium (Rb), cesium (Cs), and thallium (Tl); Z represents at least one element selected from the group consisting of boron (B), phosphorus (P), or arsenic (As) and tungsten (W). Further, a to j represent atomic ratios of the respective elements, wherein, when a=12, b=0.5 to 7, C=0 to 10, d=0 to 10 (wherein c+d=1 to 10), e=0.05 to 3, f=0 to 2, g=0.04 to 2, h=0 to 3, i=0 to 48, and j is a numerical value that satisfies the oxidation states of other elements.

The method for producing the composite oxide catalyst is not particularly limited. For example, the composite oxide catalyst may be obtained by firing a mixture obtained by mixing supply source compounds of the respective constitutional elements in an aqueous system.

Examples of the supply source compounds of the above constitutional elements include oxides, nitrates, carbonates, ammonium salts, hydroxides, carboxylates, ammonium carboxylates, ammonium halides, hydrogen acids, acetylacetonates, and alkoxides of the constitutional elements.

Examples of the supply source compound of Mo include ammonium paramolybdate, molybdenum trioxide, molybdic acid, ammonium phosphomolybdate, and phosphomolybdic acid.

Examples of the supply source compound of Fe include ferric nitrate, ferric sulfate, ferric chloride, and ferric acetate.

Examples of the supply source compound of Co include cobalt nitrate, cobalt sulfate, cobalt chloride, cobalt carbonate, and cobalt acetate.

Examples of the supply source compound of Ni include nickel nitrate, nickel sulfate, nickel chloride, nickel carbonate, and nickel acetate.

Examples of the supply source compound of Si include silica, granular silica, colloidal silica, and fumed silica.

Examples of the supply source compound of Bi include bismuth chloride, bismuth nitrate, bismuth oxide, and bismuth subcarbonate. Alternatively, it can also be supplied as a composite carbonate compound obtained by solid-dissolving the X component (one or two or more of Mg, Ca, Zn, Ce and Sm) and/or the Y component (one or two or more of Na, K, Rb, Cs and Tl) and Bi.

For example, in the case where Na is used as Y-component, a complex carbonate compound of Bi and Na may be produced by dropwise mixing an aqueous solution of a water-soluble bismuth compound such as bismuth nitrate into an aqueous solution of sodium carbonate or sodium bicarbonate, and water-washing and drying the resulting precipitate. Further, the complex carbonate compound of Bi and X-component may be produced by dropwise mixing an aqueous solution consisting of a water-soluble compound such as bismuth nitrate and a nitrate of X component with an aqueous solution of ammonium carbonate or ammonium bicarbonate, and water-washing and drying the resulting precipitate. With use of sodium carbonate or sodium bicarbonate instead of ammonium carbonate or ammonium bicarbonate, a complex carbonate compound of Bi, Na and X-component can be produced.

Examples of the supply source compound of K include potassium nitrate, potassium sulfate, potassium chloride, potassium carbonate, and potassium acetate.

Examples of the supply source compound of Rb include rubidium nitrate, rubidium sulfate, rubidium chloride, rubidium carbonate, and rubidium acetate.

Examples of the supply source compound of Cs include cesium nitrate, cesium sulfate, cesium chloride, cesium carbonate, and cesium acetate.

Examples of the supply source compound of Tl include thallium(I) nitrate, thallium(I) chloride, thallium carbonate, and thallium(I) acetate.

Examples of the supply source compound of B include borax, ammonium borate, and boric acid.

Examples of the supply source compound of P include ammonium phosphomolybdate, ammonium phosphate, phosphoric acid, and phosphorus pentoxide.

Examples of the supply source compound of As include ammonium diarseno-18-molybdate and ammonium diarseno-18-tungstate.

Examples of the supply source compound of W include ammonium paratungstate, tungsten trioxide, tungstic acid, and phosphotungstic acid.

Examples of the supply source compound of Mg include magnesium nitrate, magnesium sulfate, magnesium chloride, magnesium carbonate, and magnesium acetate.

Examples of the supply source compound of Ca include calcium nitrate, calcium sulfate, calcium chloride, calcium carbonate, and calcium acetate.

Examples of the supply source compound of Zn include zinc nitrate, zinc sulfate, zinc chloride, zinc carbonate and zinc acetate.

Examples of the supply source compound of Ce include cerium nitrate, cerium sulfate, cerium chloride, cerium carbonate, and cerium acetate.

Examples of the supply source compound of Sm include samarium nitrate, samarium sulfate, samarium chloride, samarium carbonate, and samarium acetate.

A mixture prepared by mixing the supply source compounds of respective constitutional elements in an aqueous system may be dried and then fired. The firing temperature is not particularly limited, and may be, for example, 300 to 700° C., or may be 400 to 600° C. The firing time is also not particularly limited, and may be, for example, 1 to 12 hours, or may be 4 to 8 hours.

The shape of the composite oxide catalyst is not particularly limited, and may be appropriately changed depending on the form of the reactor or the like. For example, the composite oxide catalyst may be in a granular form. In the case where the composite oxide catalyst is in a granular form, the particle size thereof may be, for example, 0.1 to 10 mm, or may be 1 to 5 mm.

Although the preferred embodiment of the present invention has been described above, the present invention is not limited to the above embodiment.

### EXAMPLES

The present invention is more specifically described with reference to Examples as follows, the present invention is not limited thereto.

### Production Example 1

### Preparation of Composite Oxide Catalyst

To 25.0 g of pure water, 12.3 g of cobalt nitrate hexahydrate and 5.8 g of iron nitrate nonahydrate were added and stirred at room temperature to be dissolved. The solution is referred to as a solution A.

Next, 1.0 g of concentrated nitric acid was added to 5.0 g of pure water to make acidic, and then 2.3 g of bismuth nitrate pentahydrate was added thereto and stirred at room temperature to be dissolved. The solution is referred to as a solution B.

Next, 10.0 g of ammonium molybdate tetrahydrate was added to 70.0 g of pure water, and stirred at room temperature to be dissolved. The solution is referred to as a solution C.

Next, the solution B was added dropwise to the solution A and mixed. The mixture solution was added dropwise to the solution C, stirred at room temperature to be mixed for 2 hours. The resulting solution was evaporated to dryness, further dried at 175° C. overnight, and then fired at 530° C. for 5 hours in an air atmosphere to obtain a composite oxide powder. The resulting powder was tablet-molded and crushed to obtain a granular solid of composite oxide catalyst having a uniform particle size of 0.85 to 1.4 mm.

### Example 1

### Preparation of Raw Material A-1

Trans-2-butene and cis-2-butene produced by Tokyo Chemical Industry Co., Ltd. were mixed at a mass ratio of 60/40 (trans-2-butene/cis-2-butene) to prepare a raw material A-1.

From the C4-hydrocarbon fraction shown in Table 1, the amount of energy required to obtain the composition of raw material A-1 was determined. Specifically, assuming a method of obtaining a straight-chain hydrocarbon through isomerization distillation of C4-hydrocarbon fraction for removal of a branched-chain hydrocarbon, the amount of input energy required for obtaining 1 kg of straight-chain butene having the same composition as in the raw material A-1 was calculated. For the calculation, VMG ver. 9.5 manufactured by Virtual Materials Group Inc., was used. The amount of input energy as a result of the calculation was as shown in Table 2.

TABLE 1

| | Composition (vol %) |
|---|---|
| Isobutane | 6.3 |
| Isobutene | 0.0 |
| 1-Butene | 40.5 |
| Butane | 27.0 |
| Trans-2-butene | 17.3 |
| Cis-2-butene | 8.9 |
| Total | 100 |

<Production of Butadiene>

A stainless steel reaction tube having an inner diameter of 10.9 mm and a length of 300 mm was filled with 11.6 mL of the composite oxide catalyst produced in Production Example 1. A thermocouple was installed in the reaction tube to measure the temperature inside the reactor. Incidentally, an electric furnace was used for the heating medium.

A mixture gas having a ratio of straight-chain butene:nitrogen:oxygen:steam=1:13.5:1.5:1.2 in the raw material gas was supplied to a preheated reactor so as to perform an oxidative dehydrogenation reaction. The gas space velocity (GHSV (h₋₁) of straight-chain butene in the raw material relative to the catalyst was set to 80 h₋₁, the average temperature in the reactor was set to 350° C., and the pressure at gauge was set to 0.0 MPa. The produced gas from the reactor outlet was sampled at 1 hour after initiation of the reaction, and analyzed by gas chromatography (Model No. 6850A manufactured by Agilent Technologies, Inc.). As a result of the analysis, the conversion rate of straight-chain butene, the selectivity of butadiene, and the yield of butadiene were as shown in Table 3.

### Example 2

### Preparation of Raw Material A-2

Trans-2-butene and cis-2-butene manufactured by Tokyo Chemical Industry Co., Ltd., were mixed at a mass ratio of 60/40 (trans-2-butene/cis-2-butene) to prepare a mixture gas.

From the C4-hydrocarbon fraction shown in Table 1, the amount of energy required to obtain the mixture gas was determined. Specifically, assuming a method of obtaining a straight-chain hydrocarbon through isomerization distillation of C4-hydrocarbon fraction for removal of a branched-chain hydrocarbon, the amount of input energy required for obtaining 1 kg of straight-chain butene having the same composition as in the mixed gas was calculated. For the calculation, VMG ver. 9.5 manufactured by Virtual Materials Group Inc., was used. The amount of input energy as a result of the calculation was as shown in Table 2.

Into a stainless steel reaction tube having an inner diameter of 10.9 mm and a length of 300 mm filled with 2.5 mL of H-type-ZSM-5 zeolite catalyst (manufactured by Tosoh Corporation, SiO₂/Al₂O₃=1900 (mol/mol)), a mixture gas having a ratio of straight-chain butene:nitrogen:oxygen:steam=1:13.5:1.5:1.2, was supplied to a preheated reactor at a gas space velocity (GHSV (h₋₁) of straight-chain butene in the raw material relative to the catalyst became 1800 h₋₁ so as to perform an isomerization reaction. Through the isomerization reaction, a raw material A-2 (trans-2-butene/cis-2-butene/1-butene=48.7/33.1/18.2) was obtained.

<Production of Butadiene>

Production of butadiene and analysis of the produced gas were performed in the same manner as in Example 1, except that the raw material A-2 was used instead of the raw material A-1 and the GHSV was changed to 100 h₋₁. As a result, the conversion rate of straight-chain butene, the selectivity of butadiene, and the yield of butadiene were as shown in Table 3.

### Example 3

### Preparation of raw material A-3

Trans-2-butene and cis-2-butene manufactured by Tokyo Chemical Industry Co., Ltd. were mixed at a mass ratio of 49.6/50.4 (trans-2-butene/cis-2-butene) to prepare a raw material A-3.

From the C4-hydrocarbon fraction shown in Table 1, the amount of energy required to obtain the composition of the raw material A-3 was determined. Specifically, assuming a method of obtaining a straight-chain hydrocarbon through isomerization distillation of C4-hydrocarbon fraction for removal of a branched-chain hydrocarbon, the amount of input energy required for obtaining 1 kg of straight-chain butene having the same composition as in the raw material A-3 was calculated. For the calculation, VMG ver. 9.5 manufactured by Virtual Materials Group Inc., was used. The amount of input energy as a result of the calculation was as shown in Table 2.

<Production of Butadiene>

Production of butadiene and analysis of the produced gas were performed in the same manner as in Example 1, except that the raw material A-3 was used instead of the raw material A-1 and the GHSV was changed to 90 h₋₁. As a result, the conversion rate of straight-chain butene, the selectivity of butadiene, and the yield of butadiene were as shown in Table 3.

### Example 4

### Preparation of Raw Material A-4

Trans-2-butene and cis-2-butene manufactured by Tokyo Chemical Industry Co., Ltd. were mixed at a mass ratio of 10.2/89.8 (trans-2-butene/cis-2-butene) to prepare a raw material A-4.

From the C4-hydrocarbon fraction shown in Table 1, the amount of energy required to obtain the composition of the raw material A-4 was determined. Specifically, assuming a method of obtaining a straight-chain hydrocarbon through isomerization distillation of C4-hydrocarbon fraction for removal of a branched-chain hydrocarbon, the amount of input energy required for obtaining 1 kg of straight-chain butene having the same composition as in the raw material A-4 was calculated. For the calculation, VMG ver. 9.5 manufactured by Virtual Materials Group Inc., was used. The amount of input energy as a result of the calculation was as shown in Table 2.

<Production of Butadiene>

Production of butadiene and analysis of the produced gas were performed in the same manner as in Example 1, except that the raw material A-4 was used instead of the raw material A-1 and the GHSV was changed to 100 h₋₁. As a result, the conversion rate of straight-chain butene, the selectivity of butadiene, and the yield of butadiene were as shown in Table 3.

### Comparative Example 1

### Preparation of Raw Material B-1

Trans-2-butene and cis-2-butene manufactured by Tokyo Chemical Industry Co., Ltd., were mixed at a mass ratio of 99.6/0.4 (trans-2-butene/cis-2-butene) to prepare a raw material B-1.

<Production of Butadiene>

Production of butadiene and analysis of the produced gas were performed in the same manner as in Example 1, except that the raw material B-1 was used instead of the raw material A-1. As a result, the conversion rate of straight-chain butene, the selectivity of butadiene, and the yield of butadiene were as shown in Table 3.

### Comparative Example 2

### Preparation of Raw Material B-2

Trans-2-butene and cis-2-butene manufactured by Tokyo Chemical Industry Co., Ltd. were mixed at a mass ratio of 73.1/26.9 (trans-2-butene/cis-2-butene) to prepare a raw material B-2.

<Production of Butadiene>

Production of butadiene and analysis of the produced gas were performed in the same manner as in Example 1, except that the raw material B-2 was used instead of the raw material A-1. As a result, the conversion rate of straight-chain butene, the selectivity of butadiene, and the yield of butadiene were as shown in Table 3.

### Comparative Example 3

### Preparation of Raw Material B-3

Trans-2-butene and cis-2-butene manufactured by Tokyo Chemical Industry Co., Ltd. were mixed at a mass ratio of 0.8/99.2 (trans-2-butene/cis-2-butene) to prepare a raw material B-3.

From the C4-hydrocarbon fraction shown in Table 1, the amount of energy required to obtain the composition of the raw material B-3 was determined. Specifically, assuming a method of obtaining a straight-chain hydrocarbon through isomerization distillation of C4-hydrocarbon fraction for removal of a branched-chain hydrocarbon, the amount of input energy required for obtaining 1 kg of straight-chain butene having the same composition as in the raw material B-3 was calculated. For the calculation, VMG ver. 9.5 manufactured by Virtual Materials Group Inc., was used. The amount of input energy as a result of the calculation was as shown in Table 2.

<Production of Butadiene>

Production of butadiene and analysis of the produced gas were performed in the same manner as in Example 1, except that the raw material B-3 was used instead of the raw material A-1. As a result, the conversion rate of straight-chain butene, the selectivity of butadiene, and the yield of butadiene were as shown in Table 3.

The raw material gas compositions and the reaction results in Examples and Comparative Examples are shown in Table 2 and Table 3. Incidentally, the raw material gas composition in Table 2 represents the proportions (mol %) of individual components in the raw material gas, and the amount of energy input represents the amount of energy required for raw material preparation (per 1 kg of 2-butene). Further, the cis-form ratio in Table 3 represents the proportion of cis-2-butene in 2-butene in the produced gas.

TABLE 2

| Raw material gas | Comparative | Comparative | | | | | Comparative |
|---|---|---|---|---|---|---|---|
| composition | Example 1 | Example 2 | Example 1 | Example 2 | Example 3 | Example 4 | Example 3 |
| Trans-2-butene | 99.6 | 73.1 | 60.0 | 48.7 | 49.6 | 10.2 | 0.8 |
| Cis-2-butene | 0.4 | 26.9 | 40.0 | 33.1 | 50.4 | 89.8 | 99.2 |
| 1-Butene | — | — | — | 18.2 | — | — | — |
| Amount of energy | — | — | 5.4 | 5.4 | 10.8 | 26.9 | 41.4 |
| input (MJ/h) | | | | | | | |

TABLE 3

| | Comparative | Comparative | | | | | Comparative |
|---|---|---|---|---|---|---|---|
| Reaction result | Example 1 | Example 2 | Example 1 | Example 2 | Example 3 | Example 4 | Example 3 |
| Butene conversion | 85.0 | 85.6 | 85.8 | 85.3 | 85.7 | 85.1 | 85.0 |
| rate (%) | | | | | | | |
| Butadiene | 72.4 | 80.6 | 84.8 | 85.5 | 84.2 | 84.9 | 85.3 |
| selectivity (%) | | | | | | | |
| Butadiene yield | 61.5 | 69.0 | 72.8 | 72.6 | 72.2 | 72.2 | 72.5 |
| (%) | | | | | | | |
| Ratio of | 19.8 | 26.5 | 30.2 | 30.8 | 32.7 | 49.4 | 56.4 |
| cis-form (mol %) | | | | | | | |

1. A method for producing butadiene, comprising:
supplying a raw material gas containing 2-butene and an oxygen-containing gas containing molecular oxygen to a reactor filled with a catalyst to obtain a produced gas containing butadiene; wherein
the catalyst contains a composite oxide containing molybdenum and bismuth; and
a proportion of cis-2-butene in 2-butene in the raw material gas is 30 to 90 mol %.

2. The method for producing butadiene according to claim 1, wherein the proportion of cis-2-butene in 2-butene in the raw material gas is 35 to 45 mol %.

3. The method for producing butadiene according to claim 1; wherein
the produced gas further contains 2-butene; and
a proportion of cis-2-butene in 2-butene in the produced gas is 28 to 50 mol %.

4. The method for producing butadiene according to claim 3, wherein the proportion of cis-2-butene in 2-butene in the produced gas is 28 to 32 mol %.

5. The method for producing butadiene according to claim 1, further comprising contacting a raw material composition containing 1-butene with an isomerization catalyst to isomerize at least a part of 1-butene to obtain 2-butene.
